(19) United States
(12) Patent Application Publication
(10) Pub. No.: US 2007/0084145 A1
(43) Pub. Date: Apr. 19, 2007

(54) PROCESS AND PACKAGING FOR A GARMENT HAVING A DESIRED STERILITY ASSURANCE LEVEL

(76) Inventor: Michael Scheerer, West Chester, PA (US)

Correspondence Address: TREXLER, BUSHNELL, GIANGIORGI,;BLACKSTONE & MARR, LTD., 105 WEST ADAMS STREET, SUITE 3600, CHICAGO, IL, 60603, US

(21) Appl. No.: 11/550,271

(22) Filed: Oct. 17, 2006

Related U.S. Application Data
(60) Provisional application No. 60/727,887, filed on Oct. 18, 2005.

(51) Int. Cl. B65B 55/02; B65B 31/00; B65B 11/58; A61L 2/08

(52) U.S. Cl. 53/425; 53/434; 53/449; 422/22

(57) ABSTRACT

A packaging for a garment and the process for forming same is disclosed. The process reduces or limits bioburden and diversity of genome on a garment. At least one non-sterile garment is placed in a heat sealable bag, a vacuum is formed in the bag and thereafter, the bag is sealed by heat sealing. The bag is then placed in a carton liner and the carton liner is closed. This defines an assembly. The assembly is placed in a carton, and the carton is closed. Thereafter, the carton containing said assembly therein is irradiated to a desired Sterility Assurance Level. All of the steps may be performed in a clean room, or the steps prior to forming the assembly are performed in the clean room and the steps after assembly are performed outside of the clean room.

This application claims the benefit of U.S. provisional application Ser. No. 60/727,887 filed on Oct. 18, 2005.

### BACKGROUND OF THE INVENTION

Sterile garments used in regulated industry environments, and garments used in non-sterile, regulated industry manufacturing environments require control of microbial levels thereon (“bioburden”). Examples of such environments are the pharmaceutical, bio-pharmaceutical, medical device and lab animal research fields. There are known biological bioburden testing procedures for such garments to ensure that restrictions are not exceeded. The garments protect the regulated industry manufacturing environments from bioburden shed by employees.

Within these regulated industry manufacturing environments, the volume of bioburden is impacted by:

1) The volume carried into the environment;

2) The rate of growth of bioburden within the environment; and

3) The cleaning protocol used to reduce or control the volume.

Additionally, the diversity of bioburden genome, such as bacteria, viruses, algae, fungus, etc., carried into these environments on garments is a considerable concern or risk.

There is a need to dramatically reduce or limit the volume of bioburden and diversity of genome entering into these environments on garments by treating the garments to kill a statistically appropriate percentage of the bioburden, and packaging the garments so that external bioburden and contamination can be kept from cross-contaminating progressively cleaner and sensitive environments.

The current techniques used today to manage bioburden volume and genome risks include the following:

1) Utilize sterile garments defined as Sterility Assurance Level (SAL) 10⁻⁶ using known sterilization techniques;

2) Autoclave all the garments on site to achieve SAL 10⁻⁶; or

3) Utilize general use garments (a general use garment being defined as woven or non-woven garments manufactured and/or processed without relevant treatment for bioburden) with high bioburden levels and control the volume with house keeping or cleaning procedures.

These options leave the customer trying to balance two extremes of cost and benefit. Options 1) and 2) are effective (statistically one or fewer colony forming unit (CFU) per 1,00,000 garments), but are very expensive and are not required for non-sterile applications. Option 3) will contribute to the environment as much as thousands of CFU per garment, resulting in very high bioburden volume contribution and unlimited risk as to the diversity of genome that can enter the environment.

### SUMMARY OF THE INVENTION

Briefly, the present invention discloses a packaging for a garment and the process for forming same. The process reduces or limits bioburden and diversity of genome on a garment. At least one non-sterile garment is placed in a heat sealable bag, a vacuum is formed in the bag and thereafter, the bag is sealed by heat sealing. The bag is then placed in a carton liner and the carton liner is closed. This defines an assembly. The assembly is placed in a carton, and the carton is closed. Thereafter, the carton containing said assembly therein is irradiated to a desired Sterility Assurance Level. All of the steps may be performed in a clean room, or the steps prior to forming the assembly are performed in the clean room and the steps after assembly are performed outside of the clean room.

### BRIEF DESCRIPTION OF THE DRAWINGS

The organization and manner of the structure and operation of the invention, together with further objects and advantages thereof, may best be understood by reference to the following description, taken in connection with the accompanying drawings, wherein like reference numerals identify like elements in which:

FIG. 1 illustrates the steps taken in a first embodiment of the process which incorporates features of the present invention;

FIG. 2 is an example of a Certificate of Irradiation/Compliance which may be used in the process of the present invention shown in FIGS. 1 or 3; and

FIG. 3 illustrates the steps taken in a second embodiment of the process which incorporates features of the present invention.

### DETAILED DESCRIPTION OF THE ILLUSTRATED EMBODIMENTS

While the invention may be susceptible to embodiment in different forms, there is shown in the drawings, and herein will be described in detail, specific embodiments with the understanding that the present disclosure is to be considered an exemplification of the principles of the invention, and is not intended to limit the invention to that as illustrated and described herein.

The present invention provides a packaging 20, 120 for items 22, 122, for example garments, and a packaging process, which dramatically reduces or limits the volume of bioburden and diversity of genome, such as bacteria, viruses, algae, fungus, etc., entering into an environment on the items 22, 122. In the present process, the items 22, 122 are packaged and then treated to kill a statistically appropriate percentage of the bioburden, so that external bioburden and contamination can be kept from cross-contaminating progressively cleaner and sensitive environments. Such an environment may be a regulated industry manufacturing environment, such as one in the pharmaceutical, bio-pharmaceutical, medical device and lab animal research fields, but uses of the present invention are not so limited. In addition, while the present disclosure describes the items 22, 122 as garments, such as hoods, shoe covers, sleeves and coveralls, the items 22, 122 to be packaged is not so limited. The present invention delivers a non-sterile Sterility Assurance Level (SAL) level that can be matched to the risk, bioburden volume control and cost objectives of the customer, thereby delivering an optimal cost-benefit relationship. The present invention also provides items 22, 122 which are stronger as less radiation is needed to achieve a desired SAL.

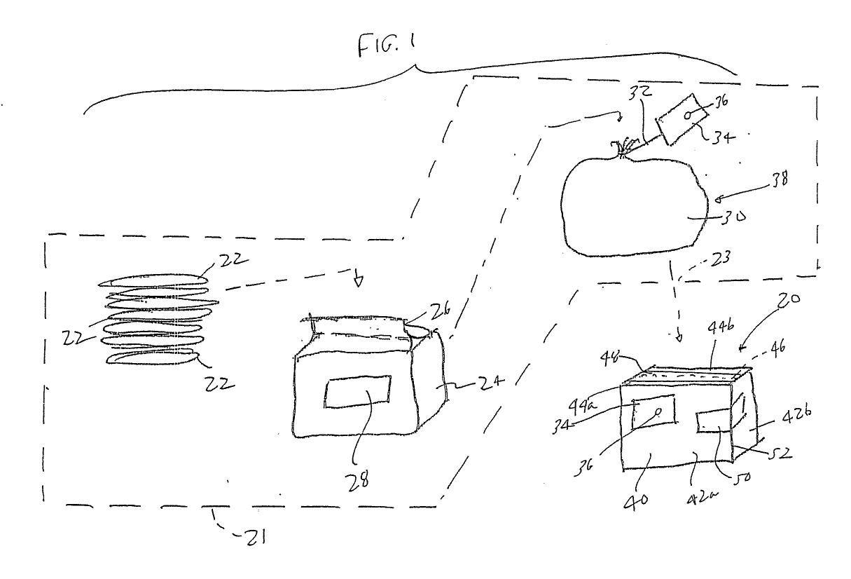

The packaging process of the present invention for forming garments 22 to be used in a non-sterile environment is shown in FIG. 1. The initial steps are performed in a clean room 21 with a positive air pressure. The clean room 21 has a pass through 23.

The non-sterile garments 22 are assembled.

The garments 22 are then placed in an open-topped bag 24, such that the garments 22 are enveloped by the bag 24. The bag 24 is formed of a suitable material that can be irradiated and heat sealed, for example polypropylene or polyethylene, and has a suitable thickness, for example 30-34 mill. Suitable means for forming a vacuum is applied to the bag 24, thereby forming a vacuum within the bag 24 and the opening in the bag 24 is sealed by a heat seal 26. A product identification label 28 is applied to the exterior of the bag 24 and affixed thereto by suitable means, such as adhesive.

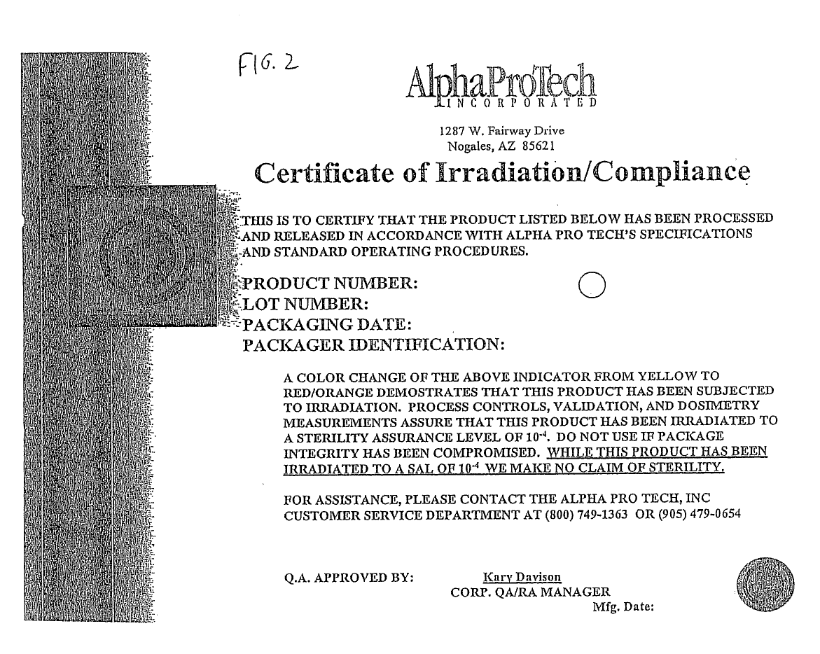

Thereafter, the bag 24 is placed into an open-topped carton liner 30, such that the bag 24 is enveloped by the carton liner 30. The carton liner 30 is formed of a suitable material that can be irradiated, for example polypropylene or polyethylene, and has a suitable thickness, for example 8 mill. The opening in the carton liner 30 is closed with a wire tie 32, and a certificate 34 of irradiation with sterilization verification dot 36 is attached to the wire tie 32 by suitable means. Such a certificate 34 is shown in FIG. 2. This combination of the carton liner 30 with the bag 24 and garments 22 therein defines an assembly 38.

Because the assembly 38 is formed in a clean room 21, the level of bioburden and particulate level on the garments 22 is minimized. The assembly 38 is then passes through the clean room 21 pass through 23 to an area outside of the clean room 21. Because of the positive air pressure in the clean room 21, contaminants from outside of the clean room 21 cannot enter into the clean room 21 through the pass through 23.

Next, in the area outside of the clean room 21, the assembly 38 is placed into a carton 40, such that the assembly 38 is enveloped by the carton 40. The carton 40 is formed of a suitable material that can be irradiated, for example corrugated cardboard having a carton thickness of 6.6 mm with a side crush strength of 500N and explosion strength of 1500 kpa. The carton 40 may be standard with four side walls 42a, 42b (only two of which are shown), a pair of top walls 44a, 44b, and a pair of bottom walls (riot shown). The top walls 44a, 44b and the bottom walls can be opened relative to the side walls to provide an opening, and can be closed to close the carton 40 and form seams 46. The carton 40 may take other forms so long as the assembly 38 is enveloped therein. All seams 46 which are formed in the closing the top walls 44a, 44b and bottom walls are sealed with tape 48. For example, the seam in the bottom walls is sealed with tape, the assembly 38 inserted therein, the top walls 44a, 44b are closed and the seam 46 is sealed with tape 48.

Thereafter, a first label 50 is applied over one corner 52 of the carton 40, such that the first label 50 is affixed to two of the side walls 42a, 42b; and a second label (now shown) is applied over an opposing corner (not shown) of the carton 40, such that the second label is affixed to the remaining two side walls (now shown). The labels 50 are affixed to the carton 40 by suitable means, such as adhesive. A certificate 34 of irradiation as shown in FIG. 2 is applied to one of the side walls, as shown 42a, of the carton 40 and affixed thereto by suitable means, such as adhesive.

Thereafter, the carton 40 containing the assembly 38 therein is irradiated by known means, for example by gamma irradiation or cobalt irradiation. The carton 40 and the assembly 38 can be irradiated to an irradiation level that best meets the cost-benefit requirements of the customer's non-sterile production environment or application. The carton 40 having the assembly 38 therein is irradiated to achieve an optimal cost-benefit non-sterile SAL of 10⁻¹ through 10⁻⁵ (sterility is defined as 10⁻⁶). The range of 10⁻⁶ through 10⁻⁵ yields a statistical probability of one or less CFU per 1,000 to 100,000 garments 22, respectively. Irradiation begins with evaluation of the items 22 and packaging materials. Because material strength is affected by irradiation, it is desirable to use the lowest dose level possible to achieve the desired SAL.

Next, the sterilization dose must be determined. This is accomplished by conducting bioburden testing of the assembly 38 to be sterilized. ISO 11137 requires that a facility conduct bioburden testing on three different manufacturing lots of the assembly 38 to be sterilized. ISO 11137 instructs the facility about whether to select a single bioburden results, or do a mathematical calculation of the results to determine the true bioburden. A qualified irradiator, which will have passed Installation Qualification (which will include equipment documentation, equipment testing, equipment calibration, irradiator dose mapping and Process Qualification where loading patterns and dose maps are conducted), is certified (all documents from the Installation Qualification are accumulated, reviewed and approved) and has Maintenance of Validation (the irradiator has a calibration program), is used to perform the irradiation. A preferred irradiation facility utilizes the latest technology in gamma irradiation, Cobalt 60, as a source with capacity of 1.5 million curies. The facility has been audited and certified by TUV, an internationally recognized institution. The certification covers Provision of Irradiation sterilization for single use medical devices in compliance with EN 552/A1:1999 and ISO 11137:1995. The facility is also EN ISO 9001:2000 certified for Quality Management System. For the Maintenance of Validation, the manufacturer also conducts irradiator re-qualification and quarterly sterilization dose auditing.

The sealing of the seams 46 on the carton 40 with tape 48 ensures that contamination cannot penetrate the carton 40 while in transit to a customer location. The packaging process allows for the carton 40 to be removed and discarded at the loading dock. At that time, the certificate 34 of irradiation is removed from the carton 40, and filed, thereby providing a critical process control step that documents that all assemblies 38 that enter the facility are irradiated at the proper non-sterile level. The assembly 38 is then stored in a clean storage area waiting to be used. When the garments 22 are needed in the dress area, the assembly 38 is loaded on to a cart and carried to the area outside the dressing area. At that time, the certificate 34 of irradiation is removed from the carton liner 30, and filed to verify that all garments 22 passing into the dress area meet the required non-sterile irradiation level. The carton liner 30 is then removed and discarded, and the sealed bag 24 is brought into the dress area. By evaluating the sealed bag 24, the operator can confirm the integrity of the garments 22. The bag 24 is then removed, and the garments 22 are placed in the appropriate bin for dispensing to employees. This packaging process and associated process of use, minimizes the risk that environmental bioburden and associated contamination migrates from one process step to the next. The SAL level can be certified using the known validation techniques as used with sterile garments. The packaging process of the present invention eliminates cross-contamination risks, while transporting the assembly 38 through progressively cleaner environments.

Because the assembly 38 is assembled in a clean room 21, less contaminants are present on the assembly 38 than if the assembly 38 were assembled in a non-clean room environment. As a result, a lower dose of radiation is required to achieve the desired SAL of 10⁻¹ through 10⁻⁵. Because less radiation is used, stronger garments 22 are provided using the present invention. In addition, irradiation facilities charge based upon the amount of radiation used. Because a lower dose of radiation is required, the cost for irradiating the assemblies 38 is reduced, thereby resulting in cost savings.

In the case of a customer using general use garments (a general use garment being defined as woven or non-woven garments manufactured and/or processed without relevant treatment for bioburden), the bioburden is reduced to SAL ¹⁰⁻⁴, and the bioburden carried into the non-sterile environment, is reduced from thousands of colony forming units (CFU) per garment, to one or less in ten thousand garments used. Because the bioburden is reduced, the cost of cleaning all surfaces in the environment, such as the walls, doors, etc., is reduced. Because cross-contamination between progressively cleaner environments is limited or eliminated as a result of the packaging process of the present invention, a lower level of risk to the environment is achieved. The cleaning cost reductions and the level of risk avoidance is achieved with 15% or less incremental cost per unit over general use garments. Some users use a sterile garment for applications that do not require a sterile garment. In this situation, the user can instead use a garment 22 packaged in accordance with the present process and the cost is reduced by approximately 40% over the cost of usage of a sterile garment, with further labor and waste stream management cost reductions resulting from the removal of unnecessary packaging and handling.

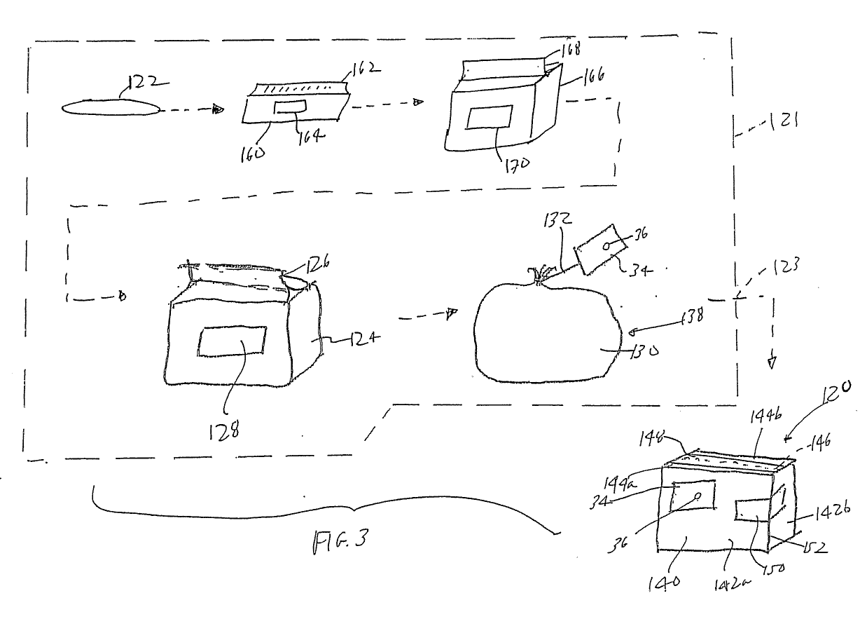

The packaging process of the present invention for forming garments 122 to be used in a sterile environment is shown in FIG. 3. The initial steps are performed in a clean room 121 with a positive air pressure. The clean room 121 has a pass through 123.

The non-sterile garments 122 are assembled.

Each garment 122 is then placed in a bag 160, such that the garment 122 is enveloped by the bag 160. The bag 160 is formed of a suitable material that can be irradiated, for example polypropylene or polyethylene, and has a suitable thickness, for example 30-34 mill. Suitable means for forming a vacuum is applied to the bag 160, thereby forming a vacuum within the bag 160, and the opening in the bag 160 is sealed by a heat seal 162. A product identification label 164 is applied to the exterior of the bag 160 and affixed thereto by suitable means, such as adhesive.

Thereafter, several of the bags 160, for example, twenty-five bags 160, are placed into an open-topped bag 166, such that the bags 160 are enveloped by the bag 166. The bag 166 is formed of a suitable material that can be irradiated, for example polypropylene or polyethylene, and has a suitable thickness, for example 30-34 mill. Suitable means for forming a vacuum is applied to the bag 166, thereby forming a vacuum within the bag 166, and the opening in the bag 166 is sealed by a heat seal 168. A product identification label 170 is applied to the exterior of the bag 166 and affixed thereto by suitable means, such as adhesive.

Thereafter, one or a plurality of the bags 166, for example, four bags 166, are placed into an open-topped bag 124, such that the one or plurality of the bags 166 are enveloped by the bag 124. The bag 124 is formed of a suitable material that can be irradiated, for example polypropylene or polyethylene, and has a suitable thickness, for example 30-34 mill. Suitable means for forming a vacuum is applied to the bag 124, thereby forming a vacuum within the bag 124, and the opening in the bag 124 is sealed by a heat seal 126. A product identification label 128 is applied to the exterior of the bag 124 and affixed thereto by suitable means, such as adhesive.

Thereafter, the bag 124 is placed into an open-topped carton liner 130, such that the bag 124 is enveloped by the carton liner 130. The carton liner 130 is formed of a suitable material that can be irradiated, for example polypropylene or polyethylene, and has a suitable thickness, for example 8 mill. The opening in the carton liner 130 is closed with a wire tie 132, and a certificate 34 of irradiation with sterilization verification dot 136 is attached to the wire tie 132 by suitable means. Such a certificate 34 is shown in FIG. 2. This combination of the carton liner 130, the bag 124, the bags 166, and the plurality of individual bags 160, each having a garment 122 therein defines an assembly 138.

Because the assembly 138 is formed in a clean room 121, the level of bioburden and particulate level on the garments 122 is minimized. The assembly 138 is then passes through the clean room 121 pass through 123 to an area outside of the clean room 121. Because of the positive air pressure in the clean room 121, contaminants from outside of the clean room 121 cannot enter into the clean room 121 through the pass through 123.

Next, in the area outside of the clean room 121, the assembly 138 is placed into a carton 140, such that the assembly 138 is enveloped by the carton 140. The carton 140 is formed of a suitable material that can be irradiated, for example corrugated cardboard having a carton thickness of 6.6 mm with a side crush strength of 500N and explosion strength of 1500 kpa. The carton 140 may be standard with four side walls 142a, 142b (only two of which are shown), a pair of top walls 144a, 144b, and a pair of bottom walls (not shown). The top walls 144a, 144b and the bottom walls can be opened relative to the side walls to provide an opening, and can be closed to close the carton 140 and form seams 146. The carton 140 may take other forms so long as the assembly 138 is enveloped therein. All seams 146 which are formed in the closing the top walls 144a, 144b and bottom walls are sealed with tape 148. For example, the seam in the bottom walls is sealed with tape, the assembly 138 inserted therein, the top walls 144a, 144b are closed and the seam 146 is sealed with tape 148.

Thereafter, a first label 150 is applied over one corner 152 of the carton 140, such that the first label 150 is affixed to two of the side walls 142a, 142b; and a second label (now shown) is applied over an opposing corner (not shown) of the carton 140, such that the second label is affixed to the remaining two side walls (now shown). The labels 150 are affixed to the carton 140 by suitable means, such as adhesive. A certificate 34 of irradiation as shown in FIG. 2 is applied to one of the side walls, as shown 142a, of the carton 140 and affixed thereto by suitable means, such as adhesive.

Thereafter, the carton 140 containing the assembly 138 therein is irradiated by known means, for example by gamma irradiation or cobalt irradiation. The carton 140 and the assembly 138 is irradiated to an irradiation level of SAL 10⁻⁶ (sterile). Irradiation begins with evaluation of the items 122 and packaging materials. Because material strength is affected by irradiation, it is desirable to use the lowest dose level possible to achieve the desired SAL.

Next, the sterilization dose must be determined. This is accomplished by conducting bioburden testing of the assembly 138 to be sterilized. ISO 11137 requires that a facility conduct bioburden testing on three different manufacturing lots of the assembly 138 to be sterilized. ISO 11137 instructs the facility about whether to select a single bioburden results, or do a mathematical calculation of the results to determine the true bioburden. A qualified irradiator, which will have passed Installation Qualification (which will include equipment documentation, equipment testing, equipment calibration, irradiator dose mapping and Process Qualification where loading patterns and dose maps are conducted), is certified (all documents from the Installation Qualification are accumulated, reviewed and approved) and has Maintenance of Validation (the irradiator has a calibration program), is used to perform the irradiation. A preferred irradiation facility utilizes the latest technology in gamma irradiation, Cobalt 60, as a source with capacity of 1.5 million curies. The facility has been audited and certified by TUV, an internationally recognized institution. The certification covers Provision of Irradiation sterilization for single use medical devices in compliance with EN 552/A1:1999 and ISO 11137:1995. The facility is also EN ISO 9001:2000 certified for Quality Management System. For the Maintenance of Validation, the manufacturer also conducts irradiator re-qualification and quarterly sterilization dose auditing.

The sealing of the seams 146 on the carton 140 with tape 148 ensures that contamination cannot penetrate the carton 140 while in transit to a customer location. The packaging process allows for the carton 140 to be removed and discarded at the loading dock. At that time, the certificate 34 of irradiation is removed from the carton 140, and filed, thereby providing a critical process control step that documents that all assemblies 138 that enter the facility are irradiated to be sterile. The assembly 138 is then stored in a clean storage area waiting to be used. When the garments 122 are needed in the dress area, the assembly 138 is loaded on to a cart and carried to the area outside the dressing area. At that time, the certificate 34 of irradiation is removed from the carton liner 130, and filed to verify that all garments 122 passing into the dress area are sterile. The carton liner 130 is then removed and discarded. The sealed bag 124 can be removed and discarded at the same time or can be left in the area outside the dressing area if all of the bags 166 are not be removed from the bag 124. A sealed bag 166 is wiped down by an operator using a sterilizing solution, such as isopropyl alcohol, and then is brought into the dress area. By evaluating the sealed bag 166, the operator can confirm the integrity of the garments 122. The bag 166 is then removed, and the garments 122 in the bags 160 are placed in the appropriate bin for dispensing to employees. Once the employee needs to use the garment 122, the bag 160 is removed and the garment 122 is donned. This packaging process and associated process of use, eliminates the risk that environmental bioburden and associated contamination migrates from one process step to the next. The SAL level can be certified using the known validation techniques. The packaging process of the present invention eliminates cross-contamination risks, while transporting the assembly 138 through progressively cleaner environments.

Because the assembly 138 is assembled in a clean room 121, less contaminants are present on the assembly 138 than if the assembly 138 were assembled in a non-clean room environment. As a result, a lower dose of radiation is required to achieve the desired SAL of 10⁻⁶ for sterilization. Because less radiation is used, stronger garments 122 are provided using the present invention. In addition, irradiation facilities charge based upon the amount of radiation used. Because a lower dose of radiation is required, the cost for irradiating the assemblies 138 is reduced, thereby resulting in cost savings.

In addition, only the sealed bag 166 is wiped down by an operator using a sterilizing solution, such as isopropyl alcohol, before being brought into the dress area. In the prior art process of sterilizing, the garments are individually packaged and each package is wiped down by an operator using a sterilizing solution, such as isopropyl alcohol, before being brought into the dress area. In the present invention, because of the packaging process, only a single bag 166 needs to be wiped down by the operator, instead of the operator being required to wipe down each bag as in the prior art. Because of human error, the possibility of more bioburden entering into the dress area is greater in the prior art, since each bag must be wiped, than in the packaging system of the present invention. In addition, the prior art process is much more time intensive as each bag must be wiped, than the present process since only a single bag needs to be wiped. As a result, the packaging system of present invention reduces the risk and the costs versus the prior art.

In this packaging process, matched sets of items 22 can be provided within the dressing area. For example, twenty-five hoods can be provided in a first bag 166 with a total of five bags 166 in a first carton 140; twenty-five pairs of shoe covers can be provided in a second bag 166 with a total of four bags 166 in a second carton 140; twenty-five pairs of sleeves can be provided in a third bag 166 with a total of six bags 166 in a third carton 140; and twenty-five coveralls can be provided in a fourth bag 166 and in a first carton 140. Therefore, the operator will always have matched sets of items 22 in the dressing area. Any extra bags 166 can be stored in the area outside of the dressing area.

If desired, the process of packaging sterile items 122 can be used package non-sterile items 22.

In each embodiment, because the bag 26, 126 is vacuum sealed, upon inspection of the bag 26, 160, 166 or 126, the user can determine if there has been a loss of integrity of the irradiation level if the vacuum is no longer present. Bioburden can migrate through very small holes, so if the vacuum is lost, there may have been a loss of integrity.

While preferred embodiments of the present invention are shown and described, it is envisioned that those skilled in the art may devise various modifications of the present invention without departing from the spirit and scope of the appended claims.

The invention claimed is:

1. A packaging process for reducing or limiting bioburden and diversity of genome on a garment comprising the steps of:
a) placing at least one non-sterile garment in a heat sealable bag through an opening in said bag;
b) forming a vacuum in said bag;
c) sealing the opening of said bag by heat sealing, such that the at least one garment is enveloped by said bag;
d) placing said bag in a carton liner;
e) closing said carton liner, such that said bag is enveloped by said carton liner and thereby defining an assembly;
f) placing said assembly in a carton,
g) closing said carton, such that said assembly is enveloped by said carton;
h) irradiating said carton containing said assembly therein, and wherein at least steps a) through e) are performed in a clean room.

2. A packaging process as defined in claim 1, wherein after step c), an identification label is applied to an exterior of said bag.

3. A packaging process as defined in claim 1, wherein steps f) through h) are performed outside of said cleanroom.

4. A packaging process as defined in claim 1, wherein said bag is formed of polypropylene or polyethylene.

5. A packaging process as defined in claim 4, wherein said bag has a thickness of 30-34 mill.

6. A packaging process as defined in claim 1, wherein said carton liner is formed of polypropylene or polyethylene.

7. A packaging process as defined in claim 6, wherein said carton liner has a thickness of 8 mill.

8. A packaging process as defined in claim 1, wherein in step d), said bag is placed through an opening in said carton liner and in step e), the opening in said carton liner is closed with a wire tie.

9. A packaging process as defined in claim 1, wherein after step e), a certificate irradiation with sterilization verification dot is attached to said carton liner.

10. A packaging process as defined in claim 1, wherein said carton is formed of cardboard.

11. A packaging process as defined in claim 10, wherein in step g), said carton is closed by adhesive tape.

12. A packaging process as defined in claim 1, wherein after step g), at least one label is applied to said carton.

13. A packaging process as defined in claim 1, wherein after step g), certificate of irradiation is applied to said carton.

14. A packaging process as defined in claim 1, wherein in step h), said carton containing said assembly therein is irradiated gamma irradiation or cobalt irradiation.

15. A packaging process as defined in claim 14, wherein said carton containing said assembly therein is irradiated to an irradiation level of Sterility Assurance Level of 10⁻¹ through 10⁻⁵.

16. A product formed by the packaging process of claim 15.

17. A packaging process as defined in claim 14, wherein said carton containing said assembly therein is irradiated to an irradiation level of Sterility Assurance Level of 10⁻⁶.

18. A product formed by the packaging process of claim 17.

19. A packaging process as defined in claim 1, wherein said carton containing said assembly therein is irradiated to an irradiation level of Sterility Assurance Level of 10⁻¹ through 10⁻⁵.

20. A product formed by the packaging process of claim 19.

21. A packaging process as defined in claim 1, wherein said carton containing said assembly therein is irradiated to an irradiation level of Sterility Assurance Level of 10⁻⁶.

22. A product formed by the packaging process of claim 21.

23. A packaging process as defined in claim 1, wherein in step a) only a single non-sterile garment is placed in said heat sealable bag, and wherein after step c) and before step d), further including the steps of:
c1) placing said bag into a second heat sealable bag through an opening in said second bag;
c2) forming a vacuum in said second bag; and
c3) sealing the opening of said second bag by heat sealing, such that the first-defined bag is enveloped by said second bag.

24. A packaging process as defined in claim 23, wherein in step c1), several bags formed in accordance with steps a) through c) are placed in said second bag.

25. A packaging process as defined in claim 24, wherein said carton containing said assembly therein is irradiated to an irradiation level of Sterility Assurance Level of 10⁻¹ through 10⁻⁵.

26. A product formed by the packaging process of claim 25.

27. A packaging process as defined in claim 24, wherein said carton containing said assembly therein is irradiated to an irradiation level of Sterility Assurance Level of 10⁻⁶.

28. A product formed by the packaging process of claim 27.

29. A packaging process as defined in claim 23, wherein after step c3) and before step d), further including the steps of:
c4) placing said second bag into a third heat sealable bag through an opening in said third bag;
c5) forming a vacuum in said third bag; and
c6) sealing the opening of said third bag by heat sealing, such that the second bag is enveloped by said third bag.

30. A packaging process as defined in claim 29, wherein in step c4), several second bags formed in accordance with steps c1) through c3) are placed in said third bag.

31. A packaging process as defined in claim 24, wherein after step c3) and before step d), further including the steps of:
c4) placing said second bag into a third heat sealable bag through an opening in said third bag;
c5) forming a vacuum in said third bag; and
c6) sealing the opening of said third bag by heat sealing, such that the second bag is enveloped by said third bag.

32. A packaging process as defined in claim 31, wherein in step c4), several second bags formed in accordance with steps c1) through c3) are placed in said third bag.

33. A packaging process as defined in claim 32, wherein said carton containing said assembly therein is irradiated to an irradiation level of Sterility Assurance Level of 10⁻¹ through 10⁻⁵.

34. A product formed by the packaging process of claim 33.

35. A packaging process as defined in claim 32, wherein said carton containing said assembly therein is irradiated to an irradiation level of Sterility Assurance Level of 10⁻⁶.

36. A product formed by the packaging process of claim 35.
